(19) United States
(12) Patent Application Publication
(10) Pub. No.: US 2013/0323166 A1
(43) Pub. Date: Dec. 5, 2013

(54) Radiation Therapy for Treating Alzheimer's Disease

(71) Applicant: William Beaumont Hospital, (US)

(72) Inventors: James Fontanesi, Bloomfield Hills, MI (US); George D. Wilson, West Bloomfield, MI (US); Alvaro A. Martinez, Bloomfield Hills, MI (US); Brian Marples, Farmington Hills, MI (US)

(21) Appl. No.: 13/790,610

(22) Filed: Mar. 8, 2013

Related U.S. Application Data
(63) Continuation-in-part of application No. PCT/US2011/050999, filed on Sep. 9, 2011.
(60) Provisional application No. 61/718,075, filed on Oct. 24, 2012.
(60) Provisional application No. 61/381,680, filed on Sep. 10, 2010.

(51) Int. Cl. A61N 5/10

(52) U.S. Cl. CPC A61N 5/1001; A61N 5/1084; A61N 2005/1087; A61N 2005/109; A61N 2005/1089
USPC 424/1.49; 600/1; 600/3

(57) ABSTRACT

A method treating dementia of the Alzheimer's type in a patient by administering ionizing radiation to the brain of the patient.

### CROSS REFERENCE TO RELATED APPLICATIONS

This application is a continuation-in-part of International Application No. PCT/US2011/050999, filed on Sep. 9, 2011, which claims priority to U.S. Ser. No. 61/381,680, filed on Sep. 10, 2010. This application also claims priority to U.S. Ser. No. 61/718,075, filed Oct. 24, 2012. The entire contents of the aforementioned applications are incorporated herein.

### BACKGROUND

Approximately 5.3 million people in the United States suffer from Alzheimer's Disease (AD) and its related diseases. AD is the seventh leading cause in death in the United States with approximately $172 billion dollars being spent on its related components on an annual basis. There are approximately 10.9 million unpaid caregivers who deal with this disease on a daily basis. The original Framingham study population was used to estimate short term (tenure), intermediate (20-30 year) and life time risk for Alzheimer's disease as well as overall risk for any dementia. In 1975 a cohort of nearly 2800 people who were 65 years of age and free of dementia provided a basis for an incident study of dementia as well as Alzheimer's disease. This cohort was followed for up to 29 years as its keen findings include significantly higher lifetime risk for both Alzheimer's and dementia in women as compared to men. For Alzheimer's, the estimated lifetime risk was nearly 1 in 5 for women compared with 1 in 10 for men.

In addition longer life expectancies and aging of baby boomers will also increase the number in percentages of Americans who will be among the oldest/old (85 years or older). Between 2010 and 2050 the oldest/old are expected to increase from 29.5 percent all older persons in the United States to 35.5 percent. Although the projected change may appear to be modest in means, there is an increase of approximately 17 million oldest-old persons, individuals who remain at high risk of developing Alzheimer's. While other major causes of death continue to experience declines those from Alzheimer's disease continue to rise. In 1991 only approximately 14,100 death certificates recorded Alzheimer's disease as an underlying cause.

AD has been linked to the following: 1) progressive amyloid deposition in various central nervous system (CNS) structures including the hippocampus, which lead to progressive memory loss especially long term; and 2) increased activity of the Tau protein thought to enhance the creation of neural tangles. Presently, efforts to slow or minimize the progressive nature of Alzheimer's Disease have met with little success. From a neurophysiological standpoint, Alzheimer's Disease appears to be a progressive process that is related to the deposition of beta amyloid and Tau protein tangles in various parts of the cortical structures of the brain including the hippocampus. Research related to gene therapy, vaccines and medical treatments have been unsuccessful at the present time at delaying the onset or progression of Alzheimer's disease.

There have been reports of the use of radiotherapy to treat tracheobronchial amyloidosis (Kurrus et al. (1998) Chest 114: 1489-1492; O'Regan et al. (2000) Medicine 79: 69-79; Kalra et al. (2001) Mayo Clin. Proc. 76: 853-856; Monroe et al. (2004) Chest 125: 784-789; Poovaneswaran et al. (2008) Medscape J. Med. 10(2): 42), localized orbital amyloidosis (Khaira et al. (2008) Orbit 27:432-437), and amyloidosis of the eyelid and conjunctiva (Pecora et al. (1982) Annals of Ophthamology 14(2): 194-196). It has also been reported that the hippocampal region has been treated with radiation in subjects suffering from mTLS (mesial temporal lobe seizures). Radiation therapy has not been used as a treatment for Alzheimer's Disease.

### SUMMARY OF INVENTION

The present invention relates to a method for treating dementia of the Alzheimer's type in a patient. The invention also relates to a method for reducing the number or size of amyloid plaques or reducing the extent of tau tangles in the brain of a patient. In either case, the method involves administering a therapeutically effective amount of ionizing radiation to the brain of the patient.

### BRIEF DESCRIPTION OF THE DRAWINGS

FIG. 1 is chart depicting the average number of amyloid-beta plaques present in the irradiated and shielded brain halves of a murine model of early-onset Alzheimer's Disease after radiation therapy treatment;

FIG. 2 is micrograph of a brain section from a mouse treated four weeks earlier with a single 15 Gy dose of radiation;

FIGS. 3A and 3B are micrographs of brain sections from mice treated with a single dose of radiation;

FIG. 4 is a series of graphs depicting the mean percent reduction in the number of plaques and the change in plaque size in the brains of mice treated with single doses of ionizing radiation; and

FIG. 5 is micrograph of a brain section from a mouse treated four weeks earlier with a 10 fractions of 1 Gy doses of radiation (fractionated radiation dosing).

FIG. 6 shows the tau-national control at 10× magnification, 20× magnification, and 40× magnification.

FIG. 7 shows the right HBRT micrograph of a brain section from a mouse treated with 1Gy×10.

FIG. 8 shows 20× magnification and 40× magnification of treated and untreated brain sections.

FIG. 9 shows 100× magnification of treated and untreated brain sections.

FIG. 10 shows a micrograph of a brain section from a mouse treated with 2 Gy×10.

FIG. 11 shows a micrograph of a brain section from a mouse.

FIG. 12 shows a graph of the overall plaque burden per mouse.

FIG. 13 shows a graph of the absolute plaque counts for mice treated with 2 Gy×10.

FIG. 14 shows a graph of the mean percent decrease in plaque counts by dose schedule.

FIG. 15 shows a graph of the treated vs. untreated hemi-brain plaque counts per dose fraction schedule.

FIG. 16 shows the results of the Alzheimer's Disease PCR Array

FIG. 17 shows the genes that were upregulated and downregulated after 24 hours.

FIG. 18 shows the genes that were upregulated and downregulated after 48 hours.

FIG. 19 shows the genes that were upregulated and downregulated after 28 days.

FIG. 20 shows the results for the immunostring test.

### DETAILED DESCRIPTION

The present invention provides for methods of treating dementia of the Alzheimer's type in a patient. The invention also provides for methods of reducing the number or size of amyloid plaques in the brain of a patient. In both cases, these methods involve administering a therapeutically effective amount of ionizing radiation to the brain of the patient.

As used herein, the term “patient” refers to a mammal who is currently experiencing the clinical symptoms of Alzheimer's Disease or who is likely to experience the clinical symptoms of Alzheimer's Disease in the future.

As used herein, the term “mammal” includes, but is not limited to, humans, gorillas, chimpanzees, orangutans, monkeys, dogs, cats, rats, mice, hamsters, gerbils, guinea pigs, rabbits, ferrets, lions, tigers, bears, zebras, giraffes, elephants, cows, horses, pigs, sheep, and goats. Preferably, the term “mammal” refers to a human.

As used herein, the term “clinical symptoms of Alzheimer's Disease” refers to those symptoms known in the art to be characteristic of dementia of the Alzheimer's type. The presence of such symptoms in a mammal may be determined by any means known in the art. For example, where the mammal is a human, the presence of such symptoms may be determined by assessing the human's degree of cognitive impairment using the Mini-Mental State Exam. A human will also be understood to have the clinical symptoms of Alzheimer's Disease if the human has been diagnosed under one or more of the following ICD-10 codes: G31.09 (other frontotemporal dementia), G30.0 (Alzheimer's disease with early onset), G30.1 (Alzheimer's disease with late onset), G30.8 (Other Alzheimer's disease), G30.9 (Alzheimer's disease, unspecified), or E85.2 (Heredofamilial amyloidosis, unspecified). However, it will be understood that a human may have the clinical symptoms of Alzheimer's Disease even if the human has not been diagnosed under one of the foregoing ICD-10 codes. The presence of the clinical symptoms of Alzheimer's Disease in a mammal may also be determined (1) by analyzing the mammal's retention of [N-methyl-¹¹C]2-(4′-methylaminophenyl)-6-hydroxybenzothiazole (the “Pittsburgh B Compound”) by positron emission tomography; (2) by analyzing the levels of certain biomarkers in the mammal's cerebrospinal fluid; or (3) by any other means known in the art.

As used herein, a mammal shall be understood to be “likely to experience the clinical symptoms of Alzheimer's disease in the future” if the mammal is identified as belonging to a familial genetic cluster having a genetic predisposition to Alzheimer's Disease. A mammal will also be understood to be “likely to experience the clinical symptoms of Alzheimer's Disease in the future” if the mammal has elevated amyloid-beta 42 or phosphorylated Tau protein levels in its cerebrospinal fluid.

As used herein, the term “therapeutically effective amount of ionizing radiation” refers to that amount of ionizing radiation that (i) treats or prevents the clinical symptoms of Alzheimer's Disease in a patient, (ii) attenuates, ameliorates, or eliminates one or more clinical symptoms of Alzheimer's Disease in a patient, (iii) prevents or delays the onset of one or more clinical symptoms of Alzheimer's Disease in a patient, (iv) inhibits, arrests the development of, or prevents the progression of one or more clinical symptoms of Alzheimer's Disease in a patient, (v) treats or prevents amyloid plaques in the brain of a patient, (vi) reduces the size of amyloid plaques or extent of tau tangles in the brain of a patient, (vii) reduces the number of amyloid plaques or extent of tau tangles in the brain of a patient, or (viii) inhibits, arrests the development of, or prevents the progression of the number of amyloid plaques or extent of tau tangles in the brain of a patient. For example, in a human or other mammal, a therapeutically effective amount can be determined experimentally in a laboratory or clinical setting, or may be the amount required by the guidelines of the United States Food and Drug Administration, or an equivalent foreign agency.

In some embodiments, treatment is defined as reducing the number of amyloid plaques in the brain. In other embodiments, treatment is defined as reducing the extent of tau tangles in the brain.

As used herein, “reducing the number of amyloid plaques” refers to a reduction of the number of amyloid plaques by more than 10%, particularly by more than 20%, particularly by more than 30%, particularly by more than 40%, particularly by more than 50%, particularly by more than 60%, particularly by more than 70%, particularly by more than 80%, particularly by more than 90%, particularly by more than 95%.

As used herein, “reducing the extent of tau tangles” or “reducing the extent of neurofibrillary tangles of tau protein” refers to a reduction of the extent of tau tangles by more than 10%, particularly by more than 20%, particularly by more than 30%, particularly by more than 40%, particularly by more than 50%, particularly by more than 60%, particularly by more than 70%, particularly by more than 80%, particularly by more than 90%, particularly by more than 95%.

The therapeutically effective amount of ionizing radiation that is administered to a subject, in the context of the present invention, should be sufficient to effect a beneficial therapeutic response in the subject over time. The dose will be determined by the efficacy of the particular radiation being employed and the condition of the subject, as well as the body weight or surface area of the subject to be treated. The amount of radiation also will be determined by the existence, nature, and extent of any adverse side-effects that accompany the administration of radiation to a particular subject.

As used herein, the term “treatment session” refers to an individual session during which a defined amount of ionizing radiation is delivered to a patient.

As used herein, the term “course of treatment” refers to one or a series of treatment sessions occurring within a discrete period of time, usually several weeks or several months. For example, a course of treatment may encompass a series of treatment sessions spanning a 2-3 week period.

As used herein, the term “total dose per treatment course” refers to the total amount of ionizing radiation, i.e., the cumulative dose of ionizing radiation, delivered to a patient during a single course of treatment. Where the course of treatment includes only one treatment session, the total dose per treatment course will be equal to the amount of ionizing radiation delivered during that treatment session. Where the course of treatment involves more than one treatment session, the total dose per treatment course will equal the sum of the amounts of ionizing radiation delivered during those treatment sessions.

As used herein, the term “total overall dose” refers to the total amount of ionizing radiation administered to a patient during that patient's lifetime. Where the patient undergoes only one course of treatment, the total overall dose will be equal to the total dose per treatment course for that course of treatment. Where the patient undergoes more than one course of treatment, the total overall dose will equal the sum of the total doses per treatment course of the courses of treatment.

The radiation may be administered externally or internally to the patient's head. Typically, ionizing radiation is subatomic particles or electromagnetic waves that are capable of detaching electrons from atoms or molecules. Ionizing radiation has wavelengths on the short end of the electromagnetic spectrum, including x-rays and gamma rays. In certain embodiments, the radiation source is a helium, carbon, hydrogen, proton, neutron, electron, or photon source. In its biological effect, electromagnetic radiation is usually considered ionizing if it has a photon energy in excess of 124 eV. X-rays are a type of electromagnetic radiation and x-rays with wavelengths of 0.1 Å correspond to a photon energy of 124 keV. Gamma rays are a type of electromagnetic radiation with a wavelength less than 10 picometers and energies typically above 100 keV. Methods and machines for administering ionizing radiation, such as x-rays and gamma rays, are known in the art.

In certain embodiments, the radiation is delivered externally as focused beam radiation. Examples of focused beam radiation include gamma knife and cyberknife. A cyberknife is a radiosurgery machine that is capable of delivering multiple beams of radiation and typically composed of a linear accelerator and a robotic arm.

The radiation may be administered internally. For example, brachytherapy may be employed to administer the radiation. Brachytherapy involves putting a radiation source inside a human at the site of treatment or within close proximity to the site of treatment. A radiation source, for example, may be placed inside a subject's brain at one or more sites, such as the hippocampus or cortical regions. The brachytherapy may be temporary or permanent brachytherapy. For temporary brachytherapy, the radiation source is put inside the body at the point of treatment for a particular amount of time and is then removed. For permanent brachytherapy, the radiation source, such a radioactive seed is put inside the body at the point of treatment and is not removed. Examples of radiation sources that may be employed in brachytherapy include, but are not limited to radioactive forms of iridium, cesium, palladium, and iodine.

In certain embodiments, radiation can be delivered by an antibody labeled with a radionuclide to deliver cytotoxic radiation to a target cell (radioimmunotherapy). In Alzheimer's Disease (AD) radioimmunotherapy, an antibody with specificity for an AD-associated antigen or components of AD such as amyloid-beta may be used to deliver a therapeutically effective amount of radiation. This may include monoclonal antibodies such as, but not restricted to, bapineuzumab or solanezumab labeled with isotopes such as, but not restricted to, iodine-131 (¹³¹I), yttrium-90 (⁹⁰Y) and Rhenium-188 (¹⁸⁸Re).

Ionizing radiation may be administered to a patient in a single course of treatment or in multiple courses of treatment. The one or more courses of treatment may take place before or after the onset of the clinical symptoms of Alzheimer's Disease in the patient. In some cases, multiple courses of treatment may be administered to the patient, beginning before the onset of the clinical symptoms of Alzheimer's Disease, and continuing after the onset of the clinical symptoms.

During each course of treatment, ionizing radiation may be administered in one or more treatment sessions. The total dose per treatment course for each course of treatment may be between about 500 and 3000 cGy. In certain embodiments, the radiation being administered is delivered in a single dose of 300-1800 cGy at 50% IDL (isodose line) by a focused based radiation source. In other embodiments, the radiation is delivered at a dose of 50 to 300 cGy per day, and preferably at a dose of 50 to 200 cGy per day, by a linear accelerator. In still other embodiments, the radiation is delivered at a dose of 50 to 600 cGy, and preferably at a dose of 50 to 200 cGy, per day by a targeted radiation source. In these latter embodiments, the course of treatment may involve an appropriate number of treatment sessions (i.e., will last an appropriate total number of days) to provide a total dose per treatment course falling within the range specified above.

In some embodiments, the course of treatment will involve a total dose per treatment course of between about 10 and 20 Gy, delivered over a period of between about one and two weeks. For example, 2 Gy of radiation may be delivered daily over a one week period (5×2 Gy). Alternatively, 1 Gy of radiation may be delivered daily over a two week period with a weekend break (10×1 Gy). Similarly, 2 Gy of radiation may be delivered daily over a two week period with a weekend break (10×2 Gy).

The radiation may be administered at a dose rate between about 50 and 1000 Monitor Units per minute, preferably between about 400 and 600 Monitor Units per minute.

The radiation may be delivered to the whole brain or regions thereof. For example the radiation may be targeted to the hippocampal region or frontal lobe of the brain.

Patients who are being treated with radiation according to the present invention may be assessed pre- and or post-treatment by neuro-cognitive testing to determine the effectiveness of the therapy. Examples of methods for screening for dementia in Alzheimer's Disease are known in the art—such as the mini-mental state examination (see e.g., Boustani et al. (2003) “Screening for Dementia”, Rockville (MD): Agency for Healthcare Research and Quality (US); 2003 U.S. Preventive Services Task Force Evidence Syntheses, formerly Systematic Evidence Reviews).

Patients who are being treated with radiation according to the present invention may also receive other treatments in combination with radiation. These may include, but not limited to, one or more of approved drugs for the treatment of Alzheimer's Disease such as the cholinesterase inhibitors (Donepezil, Rivastigmine, Tacrine and Galantamine) or glutamate modifiers such as Memantine, vaccines such as ACC-001 or AN-1792 that stimulate the body to produce its own antibodies against beta-amyloid, monoclonal antibodies such as bapineuzumab or solanezumab that target beta-amyloid, neuroactive peptides such as davunetide that cause a reduction in both amyloid peptide accumulation and tau hyperphosphorylation, intravenous immunoglobulin (IVIG) or miscellaneous agents such as reservatrol or clioquinol. In addition, radiation may be combined with one or more agents that target inflammation and the immune system using drugs that are based on immune modulation, a class that includes cytokines, lymphocyte receptors, signaling enzymes, antibodies and transcription factors. These therapeutic immune modulation drugs may include one or more of AVONEX® (interferon-beta-1a); REMICADE® (a monoclonal antibody against TNFα); ENBREL® (a fusion protein that inhibits TNF), sargramostim or molgramostim (granulocyte-macrophage colony-stimulating factor), corticosteroids such as decadron (dexamethasone) and other drugs, antibodies, vaccines and naturally occurring compounds that modulate the immune system. The treatment of subjects with radiation may also be combined with one or more agents that are known to enhance the efficacy of radiation (radiosensitizers). These agents may include one more of temozolamide, analogs of platinum (e.g., cisplatin, carboplatin, and oxaliplatin), DNA topoisomerase inhibitors (e.g., topotecan and irinotecan), antimetabolites (e.g., 5-fluorouracil, gemcitabine, etc.); epidermal growth factor receptor blockade agents (e.g.; cetuximab, getitinib, erlotinib, etc.), farnesyl transferase inhibitors, cyclo-oxygenase 2 inhibitors and agents that target vasculature (e.g., bevacizumab, thalidomide, etc.).

### EXAMPLES

### Example 1

### Single Dose Experiments in First Murine Model

Murine Model.

6 month old male B6.Cg-Tg (APPswe,PSEN1dE9)85Dbo/J (005864) mice were purchased from The Jackson Laboratory (Bar Harbor, Me.). These double transgenic mice express a chimeric mouse/human amyloid precursor protein (Mo/HuAPP695swe) and a mutant human presenilin 1 (PS1-dE9), both directed to CNS neurons. Both mutations are associated with early-onset Alzheimer's disease. Due to the ‘humanized’ Mo/HuAPP695swe transgene, the mice secrete a human amyloid-beta peptide. The animals were maintained using standard husbandry techniques familiar to one having ordinary skill in the art.

Irradiation Procedure.

At 30 weeks of age, the animals were randomized into nine groups (n=3 per group), and the right half of the brain X-irradiated at room temperature (22° C.) with a single dose of X-ray irradiation (160 kVp Faxitron X-ray machine model 43855F (0.5 mm Cu and Al filters: HVL: 0.77 (mmCU))) using a dose rate of 0.69 Gy/min. Three groups of animals received a single dose of 5 Gy, three groups received a single dose of 10 Gy, and three groups received a single dose of 15 Gy. Animals were immobilized during irradiation with ketamine (80 mg/kg), xylazine (5 mg/kg) and 0.4% isoflurane (100% O₂), or just 2-3% isofluoroane (100% O₂), as needed, to maintain treatment precision. Irradiation was limited to the right half of each animal's brain. Lead shielding was used to prevent other tissues, including the left side of the brain, from receiving any direct radiation dose. After irradiation the animals were recovered and returned to standard housing.

Tissue Harvesting.

Animals were euthanized 2, 4, or 8 weeks post treatment by CO₂ asphyxiation, with confirmation of death by cervical dislocation and decapitation. More specifically, one group of animals that received each dose was euthanized at each time point. The whole brain was harvested and fixed in 10% zinc formalin for 24 hours (Protocol, Fisher Scientific, Kalamazoo, Mich.) followed by immersion in 70% ethanol, and then subsequently paraffin embedded as is well-known to one having ordinary skill in the art. Coronal tissue sections were cut (5 μm) and mounted for antibody-specific immunohistochemistry, standard haematoxylin and eosin (H&E) for morphology and Nissl staining to assess neuronal cell density. Prior to staining, slides were deparaffinized and rehydrated through standard graded alcohol and xylene.

Staining for Amyloid-β Plaques.

Visualization of Alzheimer's plaques was accomplished by staining for amyloid-beta plaques in coronal brain sections at mid-hippocampal level (−1.70 to −1.94 mm Bregma) as described by Christensen (Christensen et al., Brain Res. 1301:116-25 2009) with several modifications. Briefly, slides were washed in tris-buffered saline (TBS) then pretreated in 88% formic acid for 3 minutes followed by an additional TBS rinse. After treatment with 0.3% H₂O₂ peroxidase block, additional binding sites were blocked using CAS block (#00-8120, Invitrogen). Sections were incubated with primary antibody (Aβ 6E10, 1:15000, mouse monoclonal, #SIG-39320, COVANCE) at room temperature for two hours then subjected to secondary antibody, polymer and DAB according to the PicTure™-MAX polymer detection kit (#87-9683, ZYMED Laboratories). Brain sections were then analyzed by light microscopy to compare the number and size of amyloid-beta plaques between the untreated left and irradiated right halves of the brain.

Quantitation of Amyloid-β Plaques and Nissl Staining.

Three stained coronal slices per mouse were analyzed to compare the number and size of amyloid-beta plaques in the irradiated versus untreated sides of the brain. Scorers were blinded to treatment dose and time point as well as to which side of the brain received radiation. Plaque counting was done at 20× magnification, with plaques less than 5 μm being excluded; anything less than this was not readily discernible at this magnification. Plaque diameter was measured at 100× magnification. Average values for each hemi-brain section were determined from measurements from two independent scorers. Hippocampus and cortex were considered separately. In addition, ten randomly selected fields within the neocortex of Nissl stained sections per brain were analyzed for cell density and radiation induced tissue damage.

Statistical Analysis.

Paired samples statistics (Student's t-test) was performed to compare the number of plaques between the irradiated and shielded sides of the brain and ANOVA to consider differences between the different doses and times post irradiation. P values of less than 0.05 were considered statistically significant.

Results of Single Dose Experiments in Murine Model.

The single dose radiation treatments were well-tolerated and no post-radiation behavioral changes were observed at any of the 2, 4, and 8 week time points, suggesting negligible or limited radiation-induced effects on normal brain tissues. In addition, histological examination of the H&E stained tissue sections indicated no evidence of a significant decrease in cell density and no compelling evidence of significant cellular necrosis. No signs of devitalization, malacia or spongiosus or classic acute or chronic inflammatory features were seen in the tissue sections, confirming that the radiation doses were insufficient to produce notable cellular effects on normal tissues at these time points. A comparison of neuronal cell density from Nissl stained brain tissue sections indicated little difference in number of neuronal cells per 200 μm×200 μm microscope field between the irradiated right-side and unirradiated left side of the brain, irrespective of dose or time post-treatment. For example, animals irradiated with 10 Gy and sacrificed 4 weeks post treatment had 28.9 (SD±16.5) and 29.5 (SD±3.2) cells in the irradiated and shielded sides of the brain, respectively. Similarly, animals irradiated with 10 Gy and sacrificed 8 weeks post treatment had 31.6 (SD±12.4) and 31.8 (SD±7.9) cells in the irradiated and shielded sides of the brain, respectively.

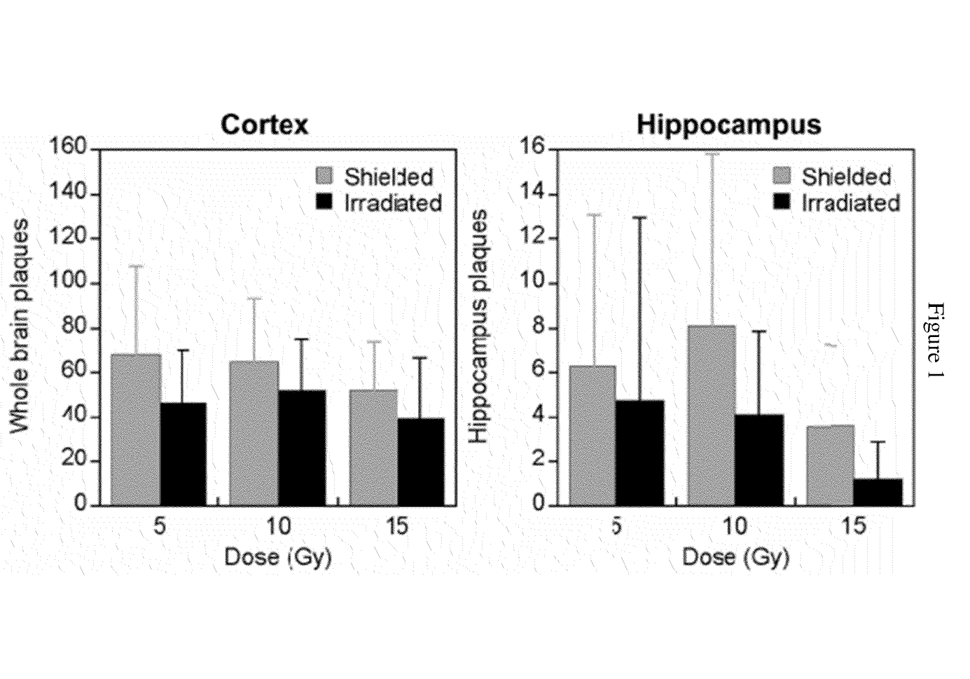

Despite animals being age-matched, the number of amyloid-6 plaques varied considerably among individual animals within each treatment group. The numerical range from the unirradiated left brain halves measured in 32, 34 and 38 week old animals was 24-136, 28-106 and 36-121 plaques, respectively. However, from 32 to 38 weeks of age the average number of plaques increased. Mean total numbers of plaques for 32, 34 and 38 week old animals were 43.9 (SD±17.6; n=9), 61.7 (SD±19.8, n=9) and 109 (SD±26.9, n=9) respectively. The mean (±SD) number of plaques in the treated and untreated halves of the cortex and hippocampus for animals irradiated with 5 Gy, 10 Gy or 15 Gy of radiation is shown in FIG. 1. For the purposes of FIG. 1, the mean number of plaques at each dose level is based on all of the animals receiving that dose level without regard for the time point at which each animal was euthanized. These data indicate that ionizing radiation leads to a reduction in plaque number. The large error bars reflect the variation in plaque number seen between individual animals irrespective of radiation treatment.

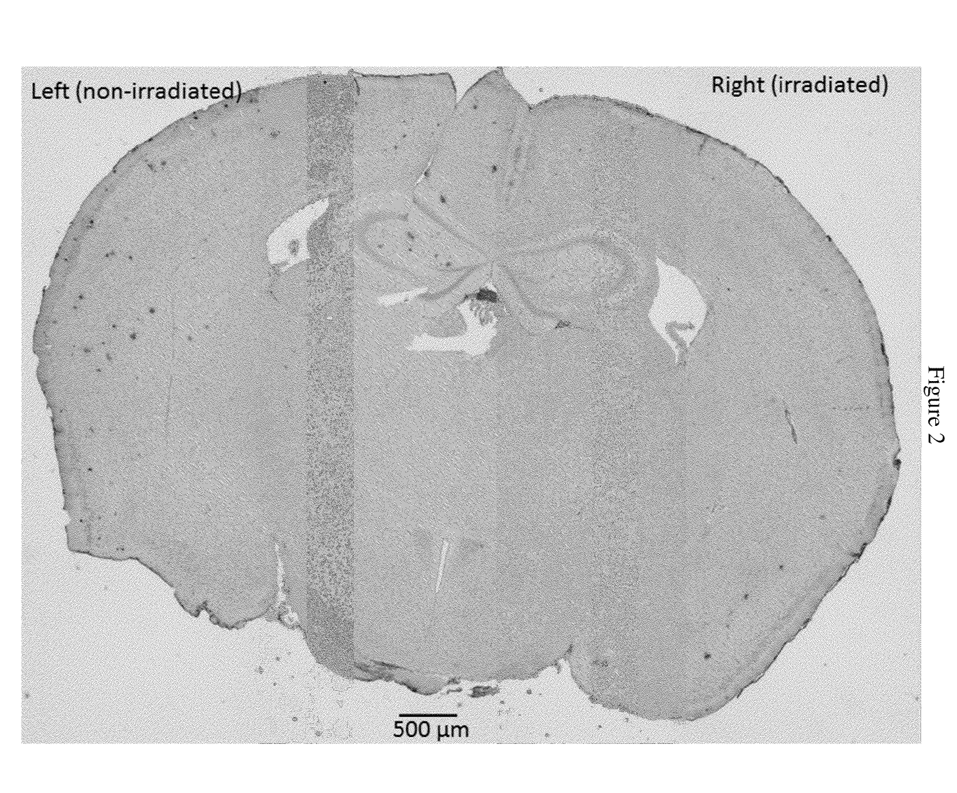

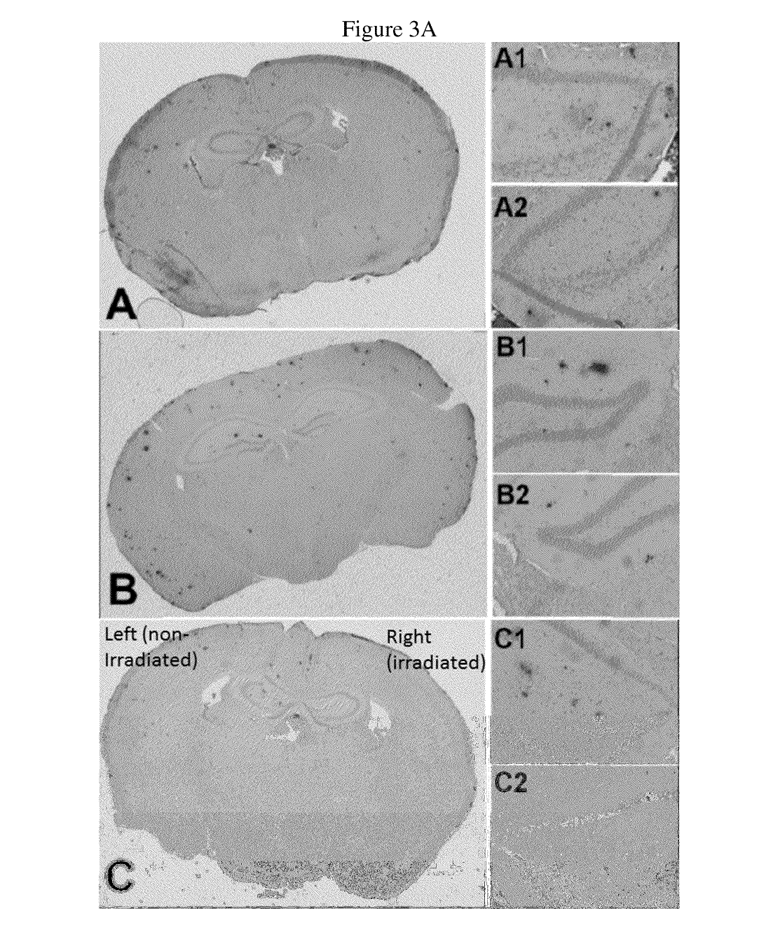

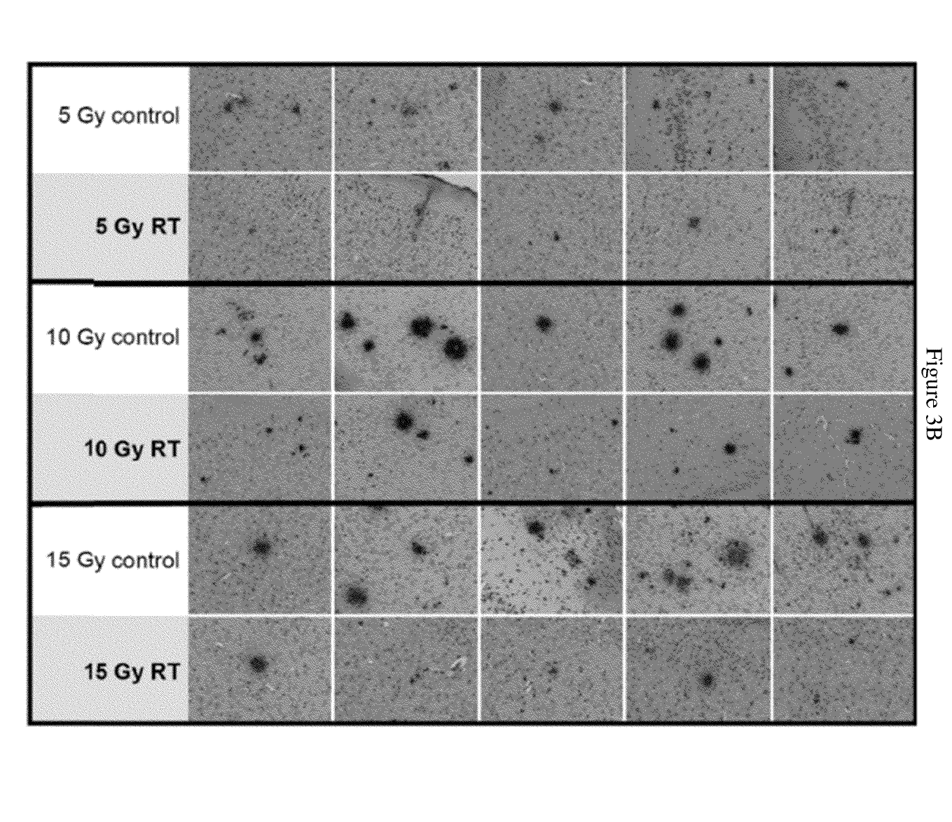

To account for the inherent variability in plaque number between individual animals within the same treatment cohort, the data analysis considered each animal serving as its own internal control. A percent change in plaque number between the irradiated and shielded side of the brain would therefore indicate the effect of the radiation treatment irrespective of the initial number of plaques. As shown in FIGS. 2 and 3A-3B, the number and size of the plaques decreased in the treated right half of the brain relative to the untreated left half of the brain.

FIG. 2 depicts a stained brain section of a mouse that was treated four weeks prior with a single dose of 15 Gy of radiation on the right side and shielded from radiation on the left side. As shown in FIG. 2, the number of plaques in the right half of the brain was significantly smaller than the number of plaques in the left half of the brain, particularly in the cortex and hippocampus regions.

FIG. 3A depicts low power micrographs of a coronal section (Bregma point −1.70) from representative animals treated with either (A) 5 Gy, (B) 10 Gy or (C) 15 Gy hemi-brain irradiation. In each case, the right side of the brain was irradiated, and the left side of the brain was shielded. The magnified panels show the hippocampus on the unirradiated (A1, B1, C1) and irradiated side (A2, B2, C2). As shown in FIG. 3A, the number of plaques was significantly smaller in the irradiated right half of the hippocampus than in the untreated left half of the hippocampus.

FIG. 3B depicts high power micrographs of representative beta-amyloid plaques from animals treated with either 5 Gy, 10 Gy or 15 Gy hemi-brain irradiation. Control images are from the shielded left side of the brain; RT signifies the irradiated right side. As shown in FIG. 3B, the diameter of the plaques was smaller in the irradiated compared with the shielded (unirradiated) brain, with an average reduction of 13.8%, 17.2% and 27.6% for animals given 5 Gy, 10 Gy and 15 Gy hemi-brain irradiation respectively.

The single dose radiation treatments, irrespective of the size of the dose, were associated with a statistically significant reduction in amyloid-β plaques throughout the brain in the irradiated side (paired t-test p=0.002). This effect was more significant when only the hippocampus region was considered (p=0.0004). There were significant differences between the observation times after radiation when all brain regions (p=0.002) or hippocampus (p=0.018) were analyzed.

Table 1 compares the mean absolute number of plaques in the irradiated right halves of the brains to the mean absolute number of plaques in the shielded left halves of the brains. The data in the table reflect the average of all animals in the study, over all time points and doses.

TABLE 1
Absolute number of plaques after single dose of irradiation in
murine model

| | | | Paired differences |
|---|---|---|---|
| | Mean | SE | Mean (±SE) |
| Plaques Hippocampus: | 6 | 1.4 | |
| Unirradiated | | | |
| Plaques Hippocampus: | 3 | 1.4 | 2.72 ± 0.79 (p = 0.0023) |
| Irradiated | | | |
| Plaques Whole Brain: | 63 | 6.54 | |
| Unirradiated | | | |
| Plaques Whole Brain: | 46 | 4.94 | 16.95 ± 4.41 (p = 0.00048) |
| Irradiated | | | |

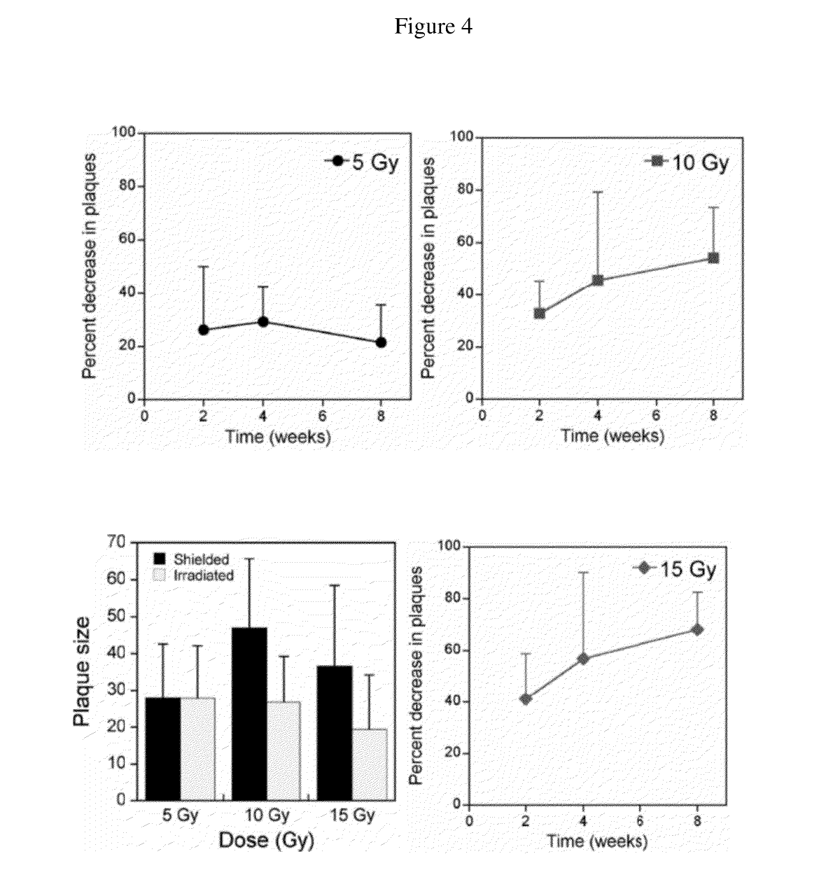

Table 2 depicts the mean percent reduction in number of plaques in the irradiated right halves of the brains, compared to the number of plaques in the shielded left halves of the brains. The values in Table 2 were determined by calculating the percentage decrease for each animal, and averaging the percentage decreases for the animals in each dose/time group (n=3 per group). Thus, the analysis accounts for the intrinsic variation in number of plaques between individual animals by using each animal as its own internal control. At 4 weeks post-treatment 5, 10 and 15 Gy doses respectively caused 29.3±13.1%, 45.7±33.6%, 56.9±33.2% reduction in plaque incidence in the whole brain. The smallest reduction in amyloid-β plaque number was seen after 5 Gy treatments, whilst comparable effects were seen for the 10 Gy and 15 Gy treatments. The data in Table 2 are depicted graphically in FIG. 4.

TABLE 2
Mean percent reduction in number of plaques in whole
brain after single dose of irradiation in murine model

| | | |
|---|---|---|
| 5 Gy | 10 Gy | 15 Gy |

| Time | Mean | SD | Mean | SD | Mean | SD |
|---|---|---|---|---|---|---|
| 2 weeks | 26.2 | 23.9 | 32.8 | 12.5 | 41.2 | 17.6 |
| 4 weeks | 29.3 | 13.1 | 45.7 | 33.6 | 56.9 | 33.2 |
| 8 weeks | 21.5 | 14.2 | 54.2 | 19.3 | 68.2 | 14.3 |

### Example 2

### Fractionated Dose Experiments in First Murine Model

Murine Model.

6 month old male B6.Cg-Tg (APPswe,PSEN1dE9)85Dbo/J (005864) mice, the same strain used in the Single Dose Experiments described in Example 1, were purchased from The Jackson Laboratory (Bar Harbor, Me.). The mice were maintained using the same techniques as described in Example 1.

Irradiation Procedure. At 30 weeks of age, the animals were randomized into two groups (n=3 per group). Both groups of animals received fractionated doses of X-ray irradiation. One group of animals received 10 doses of 1 Gy, Monday through Friday with a weekend gap. The other group received 5 doses of 2 Gy, Monday through Friday. A third group of animals received a single dose of 10 Gy, as per experiment 1. The radiation was administered at room temperature (22° C.) using a 160 kVp Faxitron X-ray machine model 43855F (0.5 mm Cu and Al filters: HVL: 0.77 (mmCU)) with a dose rate of 0.69 Gy/min. The animals were immobilized during irradiation with ketamine (80 mg/kg), xylazine (5 mg/kg) and 0.4% isoflurane (100% O₂), or 2-3% isoflurane (100% O₂), to maintain treatment precision. Irradiation was limited to the right half of each animal's brain. A lead irradiation jig was used to shield all other tissues, including the left side of the brain, from the treatment field. The first group of animals received 10×1 Gy with a 24 hour interval. The first five doses were administered over a five day period, followed by a two day gap, and then the last five doses were administered over another five day period. The second group of animals received 5×2 Gy with a 24 hour interval. The five doses were administered over 5 days. After X-irradiation, the animals were recovered and returned to standard housing. The animals were euthanized four weeks after the conclusion of the radiation treatment by CO₂ asphyxiation, with confirmation of death by cervical dislocation and decapitation. Tissue harvesting, staining, quantitation, and statistical analysis were conducted as described above in Example 1.

Results of Fractionated Dose Experiments in Murine Model.

As in the single dose experiments described in Example 1, the fractionated dose radiation treatments were well-tolerated and no post-radiation behavioral changes were observed, suggesting negligible or limited radiation-induced effects on normal brain tissues. In addition, histological examination of the H&E stained tissue sections indicated no evidence of a significant decrease in cell density and no compelling evidence of significant cellular necrosis. No signs of devitalization, malacia or spongiosus or classic acute or chronic inflammatory features were seen in the tissue sections, confirming that the radiation doses were insufficient to produce notable cellular effects on normal tissues at these time points. A comparison of neuronal cell density from Nissl stained brain tissue sections indicated little difference in number of neuronal cells per 200 μm×200 μm microscope field between the irradiated right-side and unirradiated left side of the brain, irrespective of dose or time post-treatment. For example, animals irradiated with 10×1 Gy and sacrificed 4 weeks post treatment had 38.6 (SD±9.1) and 37.8 (SD±12.5) cells in the irradiated and shielded sides of the brain, respectively. Similarly, animals irradiated with 5×2 Gy and sacrificed 4 weeks post treatment had 47.4 (SD±12.9) and 55.4 (SD±10.8) cells in the irradiated and shielded sides of the brain, respectively.

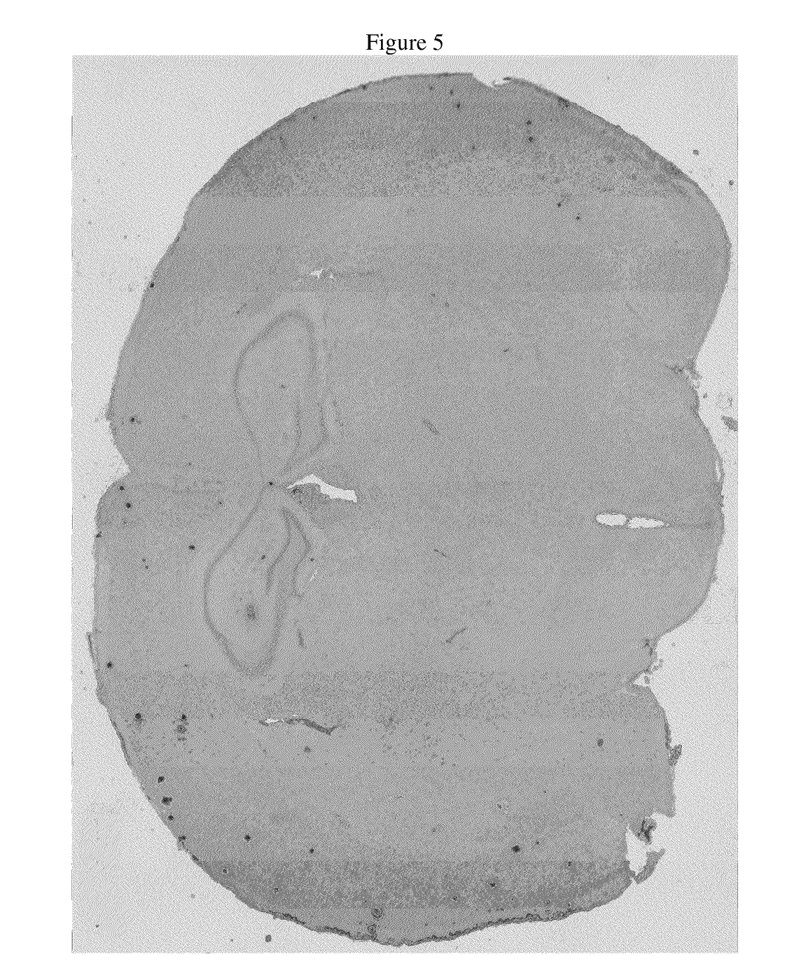

To account for the inherent variability in plaque number between individual animals within the same treatment cohort, the data analysis considered each animal serving as its own internal control. A change in plaque number between the irradiated and shielded side of the brain would therefore indicate the effect of the radiation treatment irrespective of the initial number of plaques. As shown in FIG. 5, the number and size of the plaques decreased in the treated right half of the brain relative to the untreated left half of the brain.

FIG. 5 depicts a stained brain section of a mouse that was treated four weeks prior with a 10×1 Gy fractionated dose of X-ray radiation on the right side and shielded from radiation on the left side. As shown in FIG. 5, the number of plaques in the right half of the brain was significantly smaller than the number of plaques in the left half of the brain, particularly in the cortex and hippocampus regions.

Both fractionated dose radiation treatment regimes were associated with a statistically significant (p=0.013) reduction in amyloid-(3 plaques throughout the brain in the irradiated side. The reduction in plaque incidence in the hippocampus was also statistically significant (p=0.005).

Table 3 compares the mean absolute number of plaques in the irradiated right halves of the brains to the mean absolute number of plaques in the shielded left halves of the brains.

TABLE 3
Absolute number of plaques in hippocampus and cortex at 4 weeks after
fractionated irradiation in murine model

| | Shielded | | | | Irradiated | | | |
|---|---|---|---|---|---|---|---|---|
| | Hippocampus | | Cortex | | Hippocampus | | Cortex | |
| | Mean | SD | Mean | SD | Mean | SD | Mean | SD |
| 10 × 1 Gy | 13.6 | 7.6 | 76.6 | 20.2 | 9.3 | 5.5 | 39 | 11.3 |
| 5 × 2 Gy | 10.3 | 1.5 | 81.3 | 26.5 | 4.3 | 3.2 | 51.6 | 18.3 |

Table 4 depicts the mean percent reduction in number of plaques, four weeks after the conclusion of radiation treatment, in the hippocampus region and cortex of the irradiated right halves of the brains, compared to the number of plaques in the hippocampus region and cortex of the shielded left halves of the brains. The values in Table 4 were determined by calculating the percentage decrease for each animal, and averaging the percentage decreases for the animals in each fractionated dose group (n=3 per group). Thus, the analysis accounts for the intrinsic variation in number of plaques between individual animals by using each animal as its own internal control. As shown in Table 4, the reduction in plaque incidence in the hippocampus was 76.6±48.1% after 10×1 Gy and 40.0±24.0% after 5×2 Gy. The reduction in plaque incidence in the cortex was 50.6±3.2% after 10×1 Gy and 71.8±38.4% after 5×2 Gy. By comparison, the reduction in plaque incidence in the hippocampus was 55±12% after a single 10 Gy dose. Radiation treatment also significantly reduced the mean size of Aβ plaques from 48.7±18 μM to 26±12 μM (10×1 Gy).

TABLE 4
Mean percent reduction in number of plaques in hippocampus and cortex
after fractionated dose irradiation in murine model

| | |
|---|---|
| 10 × 1 Gy | 5 × 2 Gy |

| Brain Region | Mean | SD | Mean | SD |
|---|---|---|---|---|
| Hippocampus | 76.6 | 48.1 | 40.0 | 24.0 |
| Cortex | 50.6 | 3.2 | 71.8 | 38.4 |

### Example 3

### Low Dose Fractionated Hemi Brain Irradiation in Second Transgenic AD Mouse Models: Impact on Neurofibrillary Tangles of Tau Protein

This experiment used a different mouse model of Alzheimer's Disease that has genetic modifications that produce early onset Alzheimer's Disease. The model is a triple transgenic mouse strain with the amyloid beta plaque formation and also excess neurofibrillary tangles of tau protein. Modest does radiation therapy treatments reduced the extent of neurofibrillary tangles of tau in this mouse strain, suggesting that radiation therapy is a treatment for Alzheimer's disease.

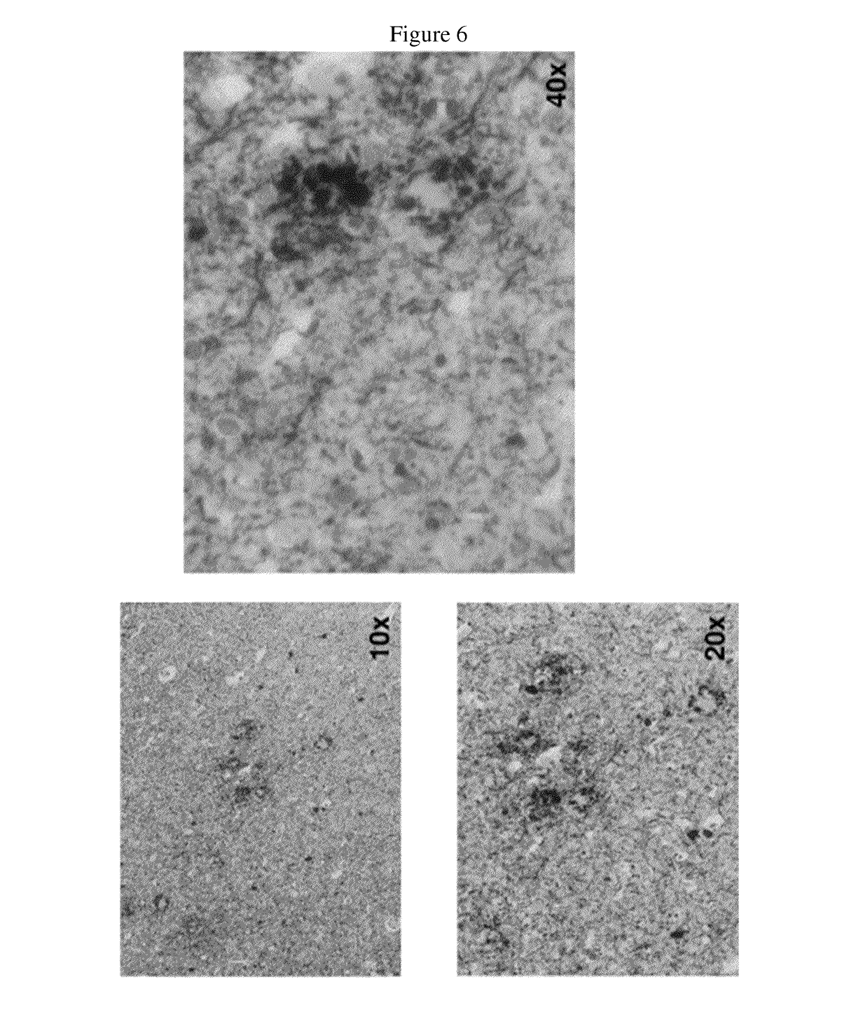

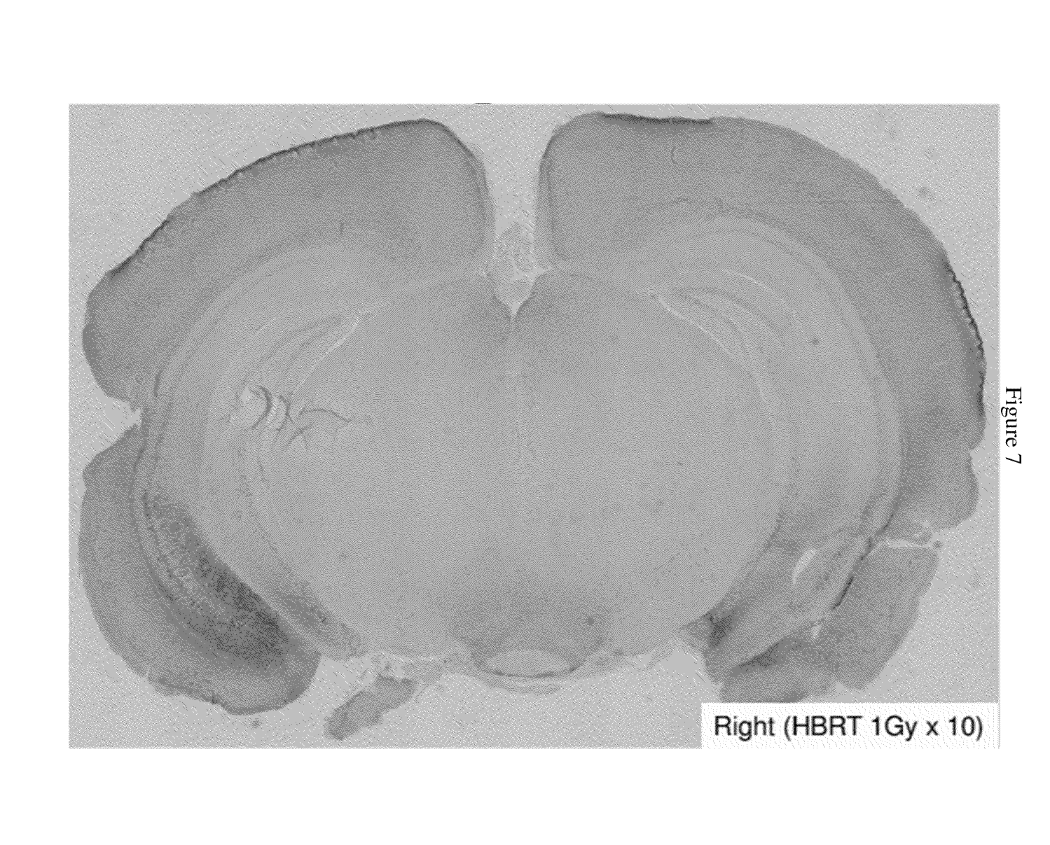

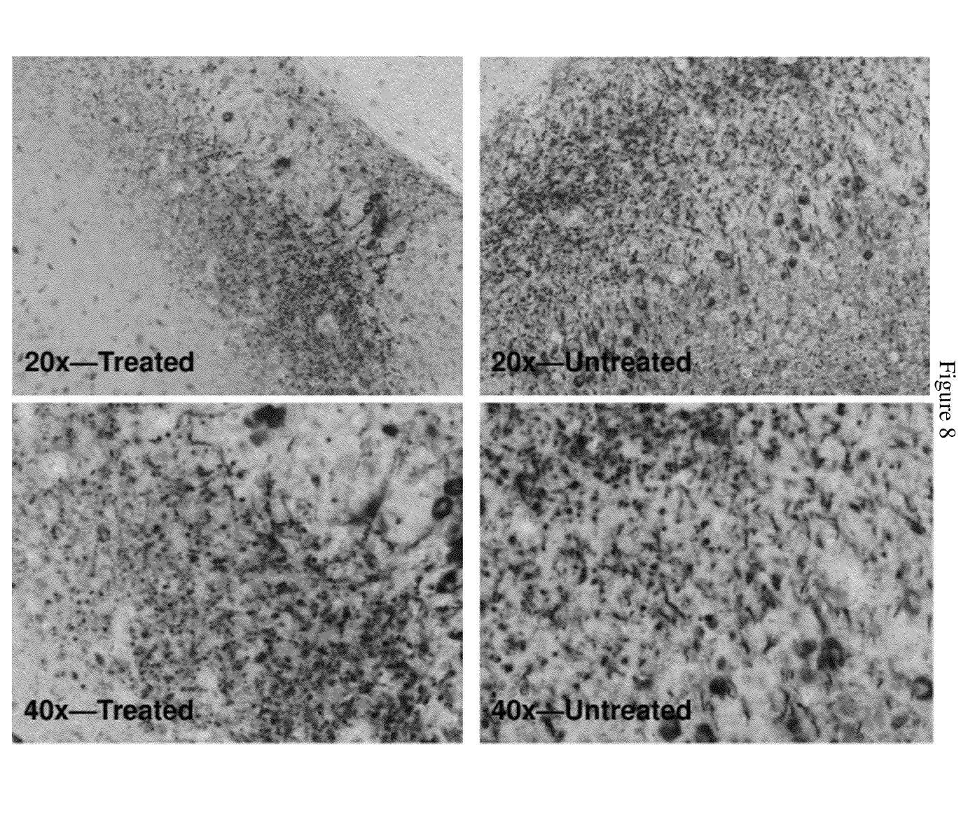

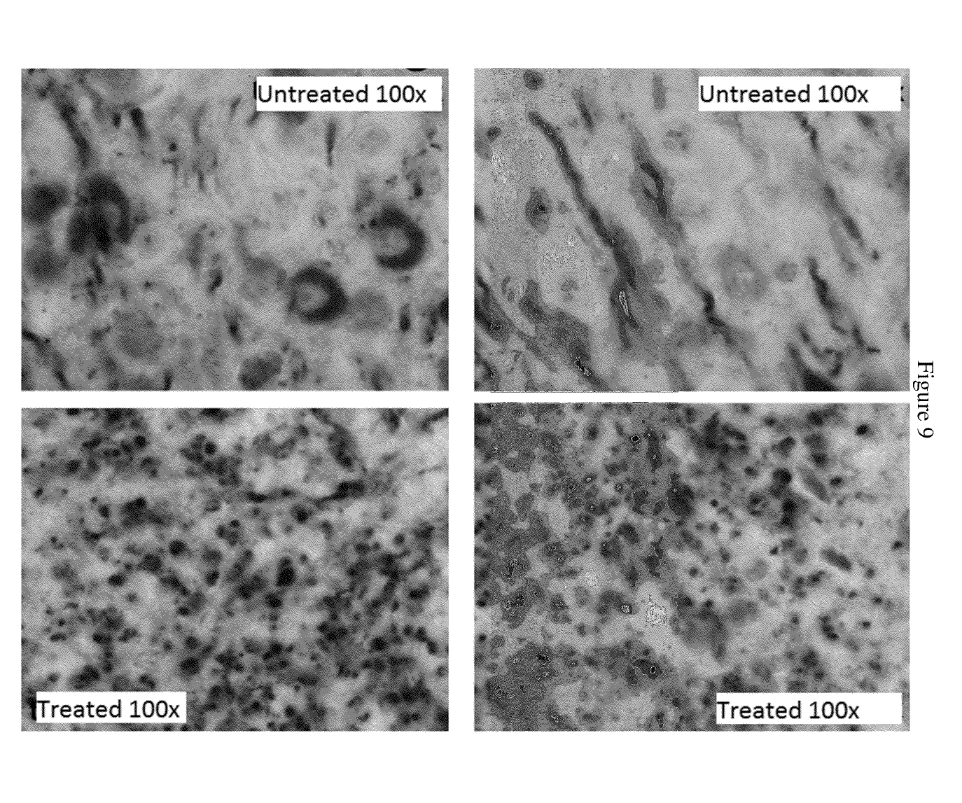

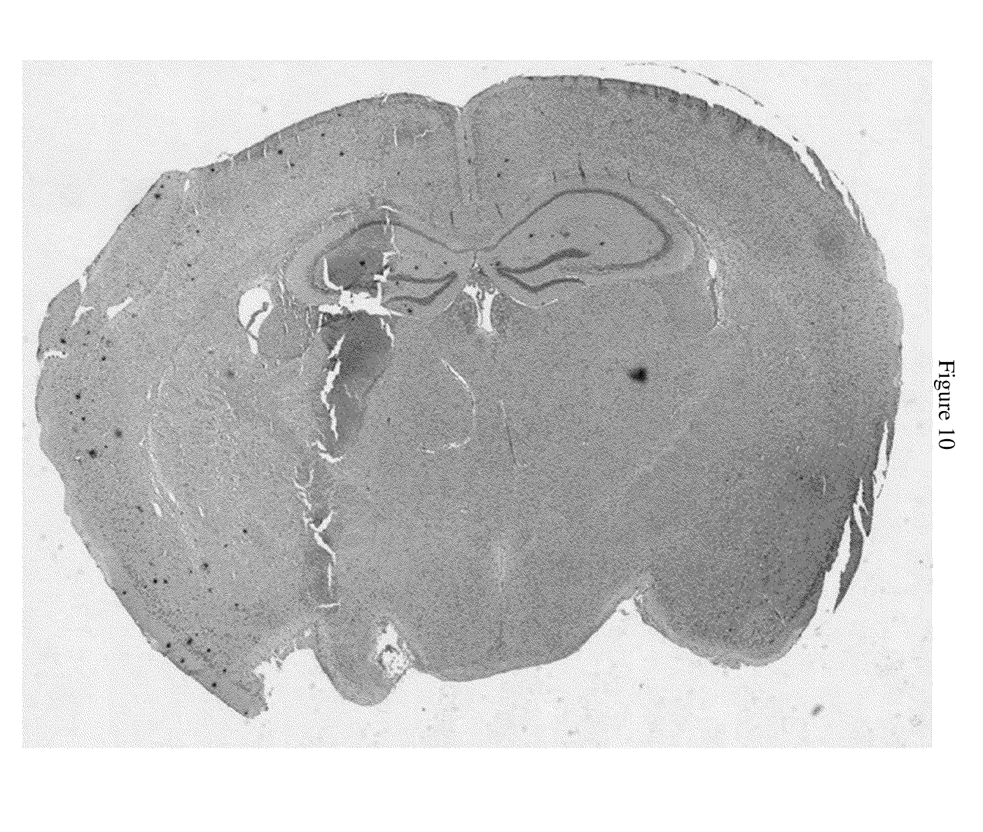

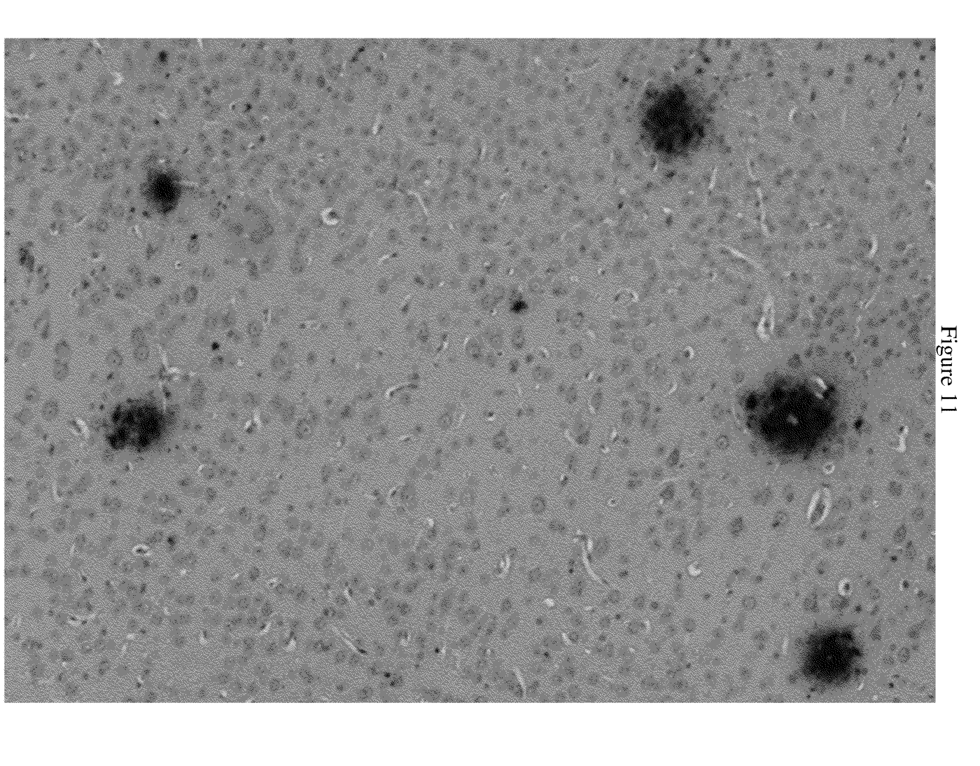

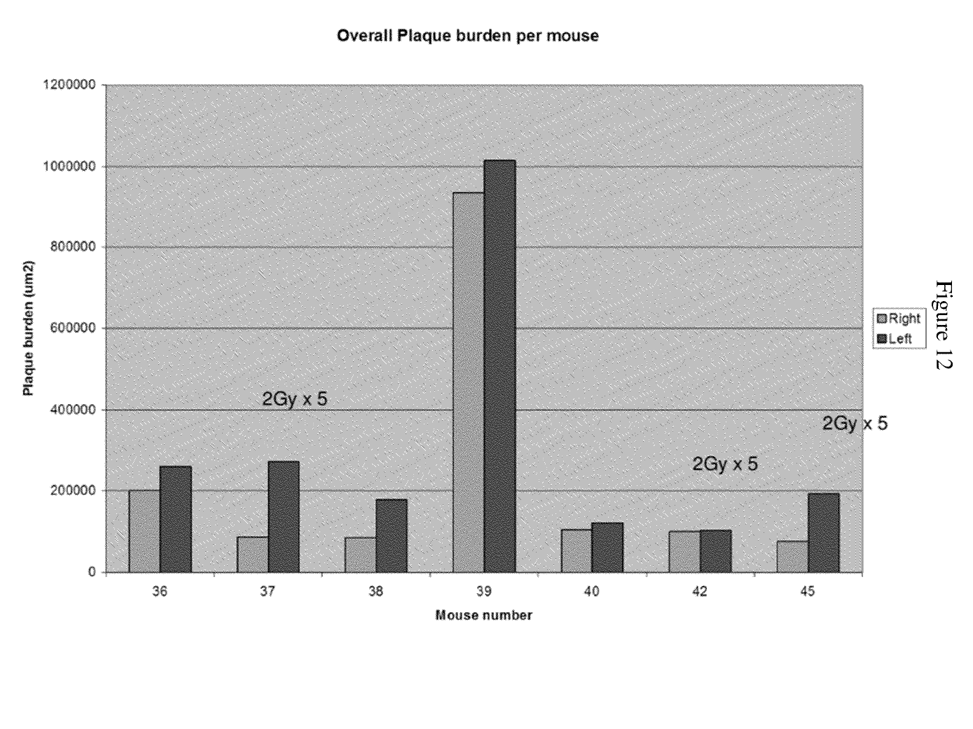

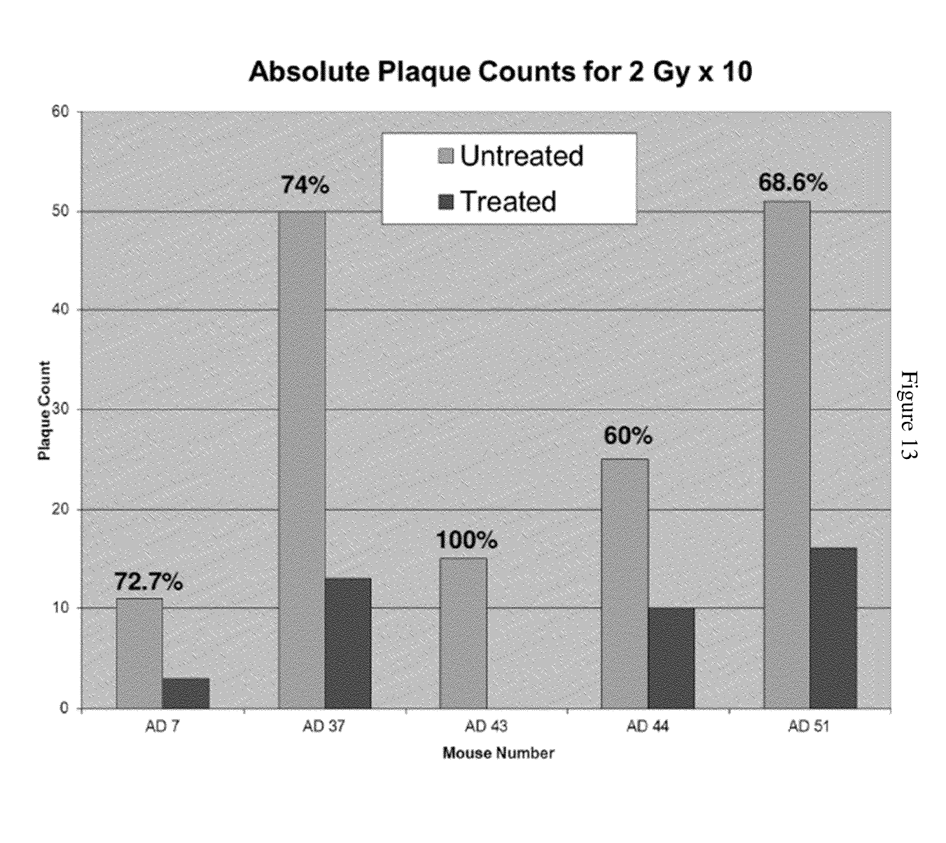

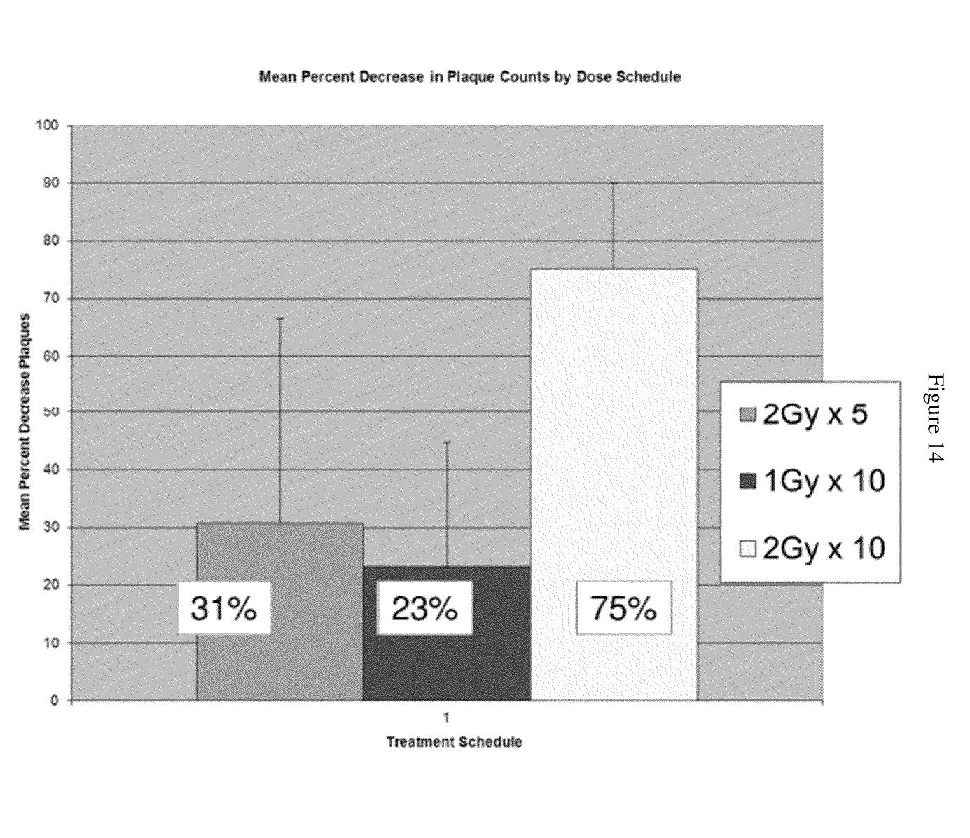

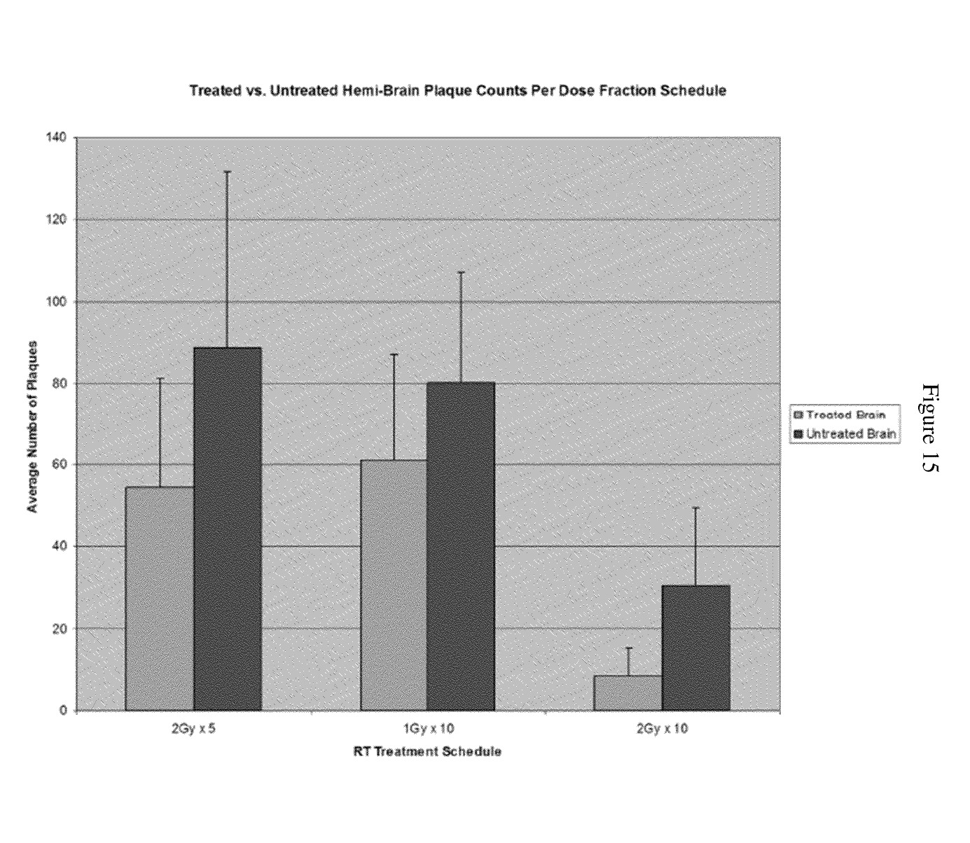

FIGS. 6-15 show the results of this mouse model. The genes associated with Alzheimer's Disease are shown in the following table:

| | |
|---|---|
| Beta-Amyloid Generation, Oligomerization, | Secretases: Adam9, Aph1a, Bace1, Bace2, |
| Clearance, and Degradation | Ctsb, Ncstn, Psen1, Psen2 |
| | Other Peptidases Involved in Beta-Amylold |
| | Degradation: Ide, Plat, Plau, Plg |
| | Beta-Amyloid Clearance Throuoh Endocytosis: |
| | Aplp1, App, Lrp1, Lrp6, Lrp8 |
| | Other Genes Involved In Beta-Amyloid |
| | Metabolism: A2m, Ache, Apbb1, Apbb2, Apoe, |
| | Bche, Ubqln1 |
| Microtubule and Cytoskeleton | Apoe, Mapt, Mtap2, Pkp4, Prkci |
| Reorganization | |
| Synaptic Formation | Synaptic Transmission: Apba1, Apoe, Chat. |
| | Other Synaptic Functions: Ache, Apba1, Apbb1, |
| | Apbb2, Apoe, Bdnf |
| Cholesterol Metabolism | Abca1, Apoa1, Apoe, Lrp8 |
| Lipid and Lipoprotein Metabolism | Appa1, Apoe, Clu, Hsd17b10, Igf2, Lpl, Lrp1, |
| | Lrp8, Sncb |
| Hormone and Hormone Processing | Bace2, Igf2 |
| Apoptosis | Induction of Apoptosis: Apoe, Casp3, Casp4, |
| | Ern1, Prkca, Prkce |
| | Anti-Apoptosis: Il1a, Mpo, Prkcz, Psen1, Snca |
| | Other Genes Involved in Apoptosis and Cell |
| | Death: Aplp1, App, Clu, Ep300, Mapt, Nae1, |
| | Psen2 |
| Cell Cycle Regulators | Cell Cycle Arrest: Apbb1, Apbb2, Ern1. |
| | Other Cell Cycle Genes: Apbb1, Apbb2, Cdk1, |
| | Cdk5, Cdkl1, Nae1, Ep300, Il1a, Prkca |
| Protein Kinases | Cdk1, Cdk5, Cdkl1, Ern1, Gsk3a, Gsk3b, Insr, |
| | Prkca, Prkcb, Prkcc, Prkcd, Prkce, Prkci, Prkcq, |
| | Prkcz |
| Cell Signaling Molecules | Wnt Receptor Signaling: Gsk3b, Lrp6. |
| | Notch Signaling: Aph1a, Ncstn, Psen1, Psen2 |
| | G-Protein Coupled Receptor Signaling: Aplp2, |
| | Gnao1, Gnaz, Gnb1, Gnb2, Gnb4, Gnb5, |
| | Gng10, Gng11, Gng3, Gng4, Gng5, Gng7, |
| | Gng8, Gngt1, Gngt2 |
| | Intracellular Signaling: Apba3, Apbb2, Prkca, |
| | Prkcb, Prkcc, Prkcd, Prkce, Prkci, Prkcq, Prkcz, |
| | Psen1, Psen2 |
| | Other Signaling Molecules: Gap43, Gnb2, Ide, |
| | Il1a, Insr, Nae1, Plau |
| Transcription regulators | Apbb1, Apbb2, Ep300, Ern1 |
| Other Genes Involved in Alzheimer's disease | Oxidoreductases and Oxidative Stress: |
| | Hsd17b10, Mpo, Uqcrc1, Uqcrc2 |
| | Proteases: Ctsc, Ctsd, Ctsg, Ctsl, Uqcrc2 |
| | Protease Inhibitors: Aplp2, App, Serpina3c |

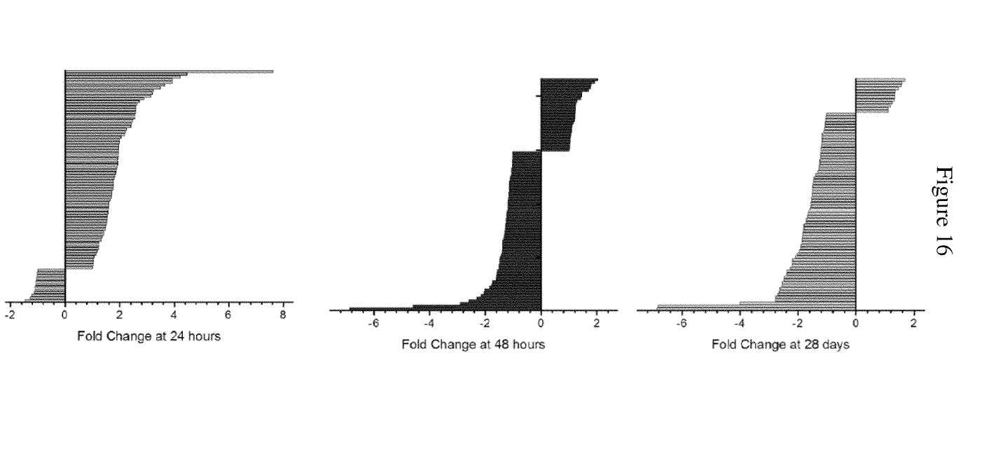

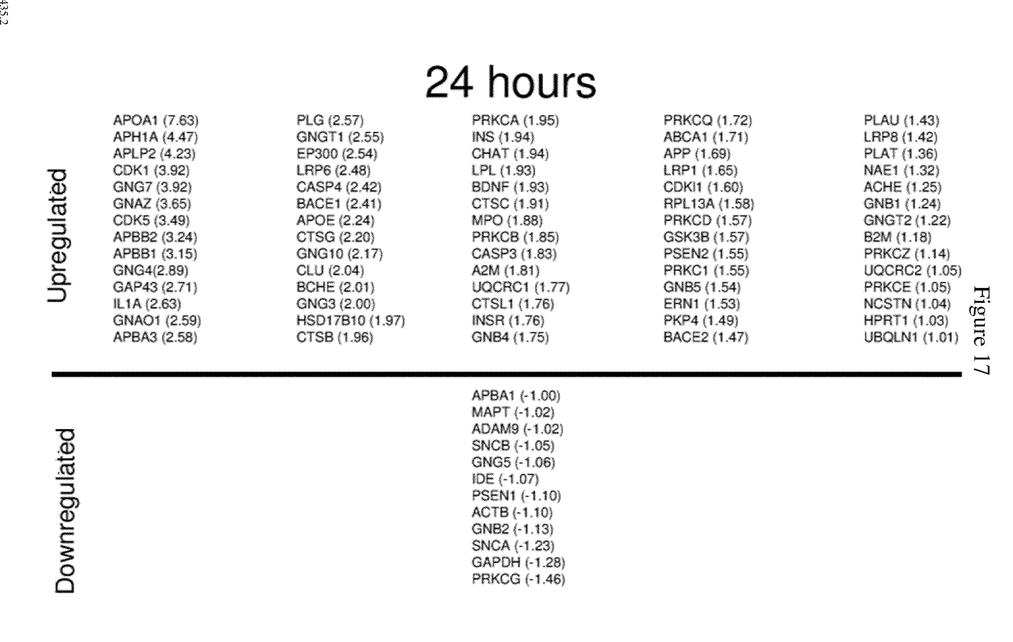

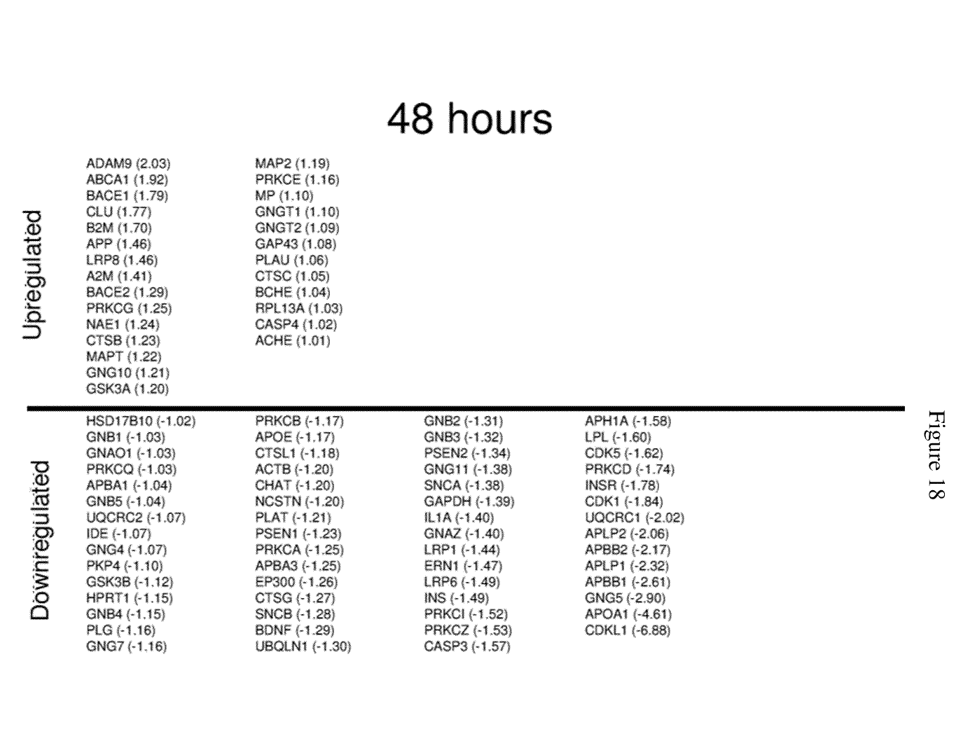

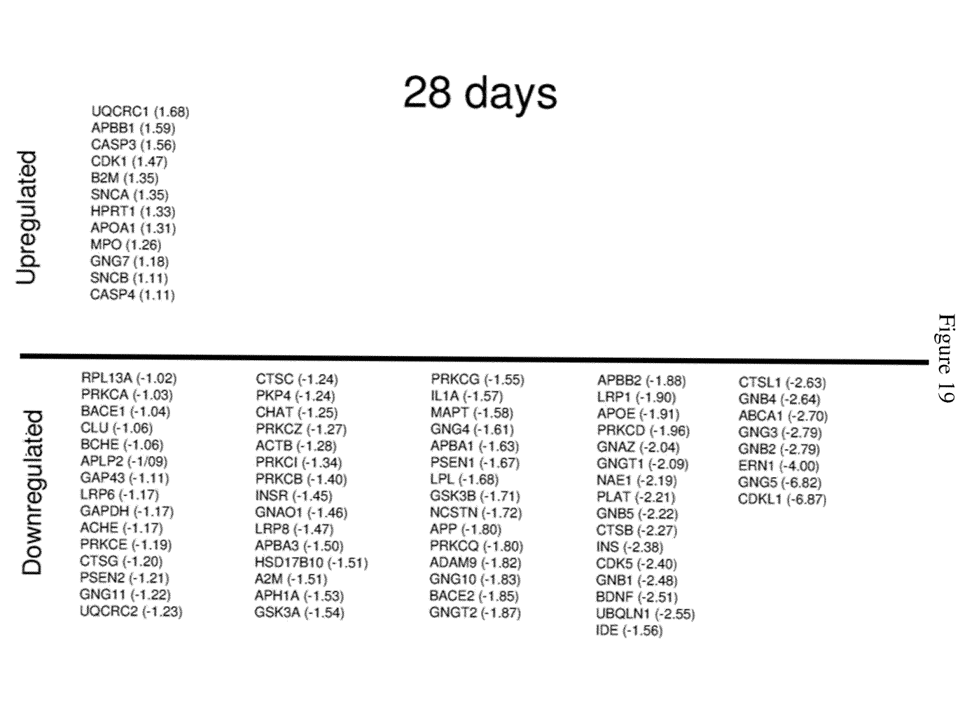

An Alzheimer's Disease RT2 Profiler™ PCR from SABiosciences is shown in FIG. 16. There was a significant decrease in presenilin 1 at 48 hours (p=0.014). There was a significant increase β-site APP-cleaving enzyme 2 (p=0.01). Amyloid β (A4) precursor like protein 1 (APLP1), APLP2, and apolipoprotein A-1 had a 2-4 fold decrease in expression at 48 hours. The results are shown in FIGS. 17-19.

The following genes were included in the cytokine array:

| | |
|---|---|
| Chemokine Genes | Ccl1, Ccl11, Ccl12, Ccl17, Ccl19, Ccl2, Ccl20, |
| | Ccl22, Ccl24, Ccl25, Ccl3, Ccl4, Ccl5, Ccl6, |
| | Ccl7, Ccl8, Ccl9, Cx3cl1, Cxcl1, Cxcl10, Cxcl11, |
| | Cxcl12 (Sdf1), Cxcl13, Cxcl15, Pf4, Cxcl5, |
| | Cxcl9, Il13. |
| Chemokine | Ccr1, Ccr2, Ccr3, Ccr4, Ccr5, Ccr6, Ccr7, Ccr8, |
| Receptors | Ccr9, Ccr10, Cxcr3, Cxcr2, Xcr1. |
| Cytokine Genes | Ifng (IFNy), Il10, Il11, Il13, Il15, Il16, Il17b, Il18, |
| | Il1a, Il1b, Il1f6, Il1f8, Il20, Il3, Il4, Itgam, Itgb2, |
| | Lta, Ltb, Mif, Aimp1, Spp1, Tgfb1, Tnf, Cd40lg |
| Cytokine Receptors | Ifng (IFNy), Il10ra, Il10rb, Il13, Il13ra1, Il1r1, |
| | Il1r2, Il2rb, Il2rg, Il5ra, Il6ra, Il6st, Tnfrsf1a |
| | (TNFR1), Tnfrsf1b (TNFR2) |
| Other Genes | Abcf1, Bcl6, Cxcr5, C3, Casp1, Crp, Il1r1, |
| Involved in | Cxcr2, Tollip |
| Inflammatory | |
| Response | |

AD and cytokine multiplex arrays were conducted using soluble cytokines and Millipore Multiplex_{MAP}. The following genes were examined: IL-1α, IL-1β, IL4, IL6, IL10, CSF3 (G-CSF), CSF2 (GM-CSF), CSF1 (M-CSF), TNF-α, IFN-γ, MIP-1α, MCP-1 and MIP. The results for the cytokine array are summarized in the following table:

| | |
|---|---|
| MCP1: monocyte chemotactic | biomarker to monitor the inflammatory |
| protein 1 | process of AD |
| | 5 × 2 Gy at 4 weeks (p = 0.047) |
| MIP-1α: macrophage | Elevated in AD vessels and is regulated |
| inflammatory protein 1-alpha | by oxidative stress |
| | 5 × 2 Gy at 4 weeks (p = 0.05) |
| G-CSF: granulocyte-colony | novel biomarkers for early detection of |
| stimulating factor | clinical Alzheimer's disease PLoS One. |
| | 2011; 6 (3) |
| | 5 × 2 Gy at 4 weeks (p = 0.08) |

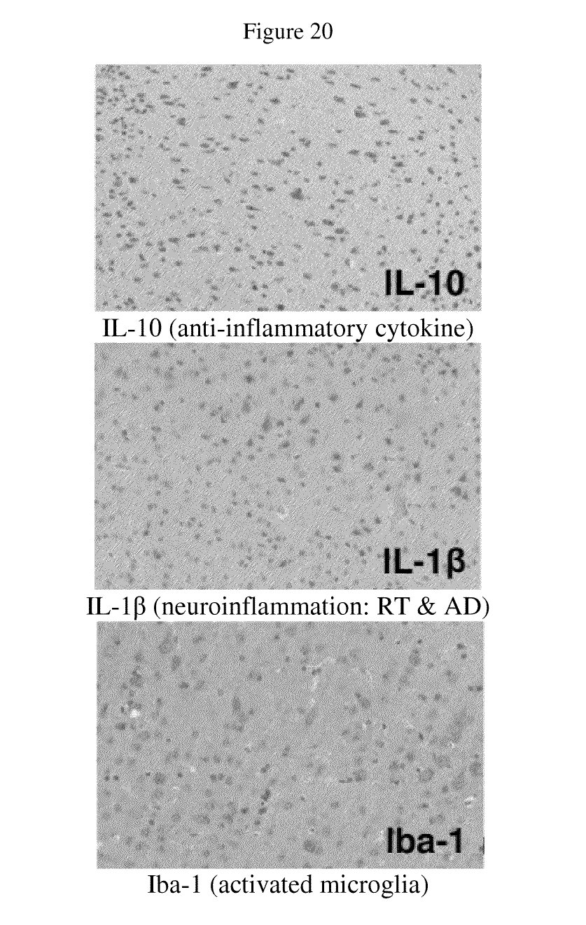

FIG. 20 shows the results for the immunostring test. The following results were obtained:

| | |
|---|---|
| IL-10 (anti-inflammatory | Trend percent positive IL-10 staining |
| cytokine) | cells in irradiated vs. shielded hemi- |
| | brains (p = 0.07) |
| | 1 Gy × 10 > 2 Gy × 5 (p = 0.00145) |
| IL-1β (neuroinflammation: | Increased expression after RT |
| RT & AD) | 2 Gy × 5: 24-48 hrs 28.5% positive cells |
| | vs. 33.2% at 28 days |
| Iba-1 (activated microglia) | Increased expression after RT |
| | 2Gy × 5: 33.5% at 24 h vs 54% at 28 d |
| | (p = 0.001) |

### Example 4

### Early Low Dose CNS Irradiation in Young Transgenic Mice

Objectives.

To determine the effects of low dose fractionated hemi brain irradiation on the eventual development of amyloid plaques in a transgenic mouse model.

Methods.

A transgenic mouse model was irradiated at 10 weeks, which is the time when amyloid initially develops. In one experiment, the mice were treated using a 5×200 cGy regimen in the whole brain. In other experiments, the mice were treated using both a 5×200 cGy and a 10×200 cGy regimen in the hemi brain. At 7 months, the animals were sacrificed, and coronal sections were stained for H&E, anti-beta amyloid, and IL-10. Plaque assessment was completed using published techniques and were compared to mice which received similar treatment at 6 months and sacrificed one month later.

Results. There was a statistical reduction in amyloid number and volume in the younger treated hemi brain sections (p=0.0004). When compared to mice treated at 6 months, there were noted statistical reductions in plaque number in both treated (p=0.01) and shielded (p=0.03) sides. There was also a trend towards significance for IL-10 staining in the irradiated tissues.

Conclusions.

This data demonstrates that the use of early low dose fractionated irradiation can potentially reduce the development of amyloid plaques in younger transgenic mice. The increased IL-10 staining also suggests a CNS inflammatory cell mediated pathway activation.

### Example 5

### Effect of Ionizing Radiation on Cognitive Impairment in Murine Model

Murine Model.

6 month old male B6.Cg-Tg (APPswe,PSEN1dE9)85Dbo/J (005864) mice, the same strain used in Examples 1 and 2, will be purchased from The Jackson Laboratory (Bar Harbor, Me.). The mice will be maintained using the same techniques as described in Examples 1 and 2.

Pre-Irradiation Cognitive Testing.

Prior to the delivery of ionizing radiation the animals will be tested with the Morris Water Maze and Barnes Maze to assess spatial learning and memory. The procedures for cognitive testing using the Morris Water Maze and Barnes Maze are well known in the art.

Irradiation Procedure.

The animals will be randomized into seven groups (n=3 per group). Ionizing radiation will be delivered to the brains of the animals in each group. The radiation will be administered at room temperature (22° C.) using a 160 kVp Faxitron X-ray machine model 43855F (0.5 mm Cu and Al filters: HVL: 0.77 (mmCU)) with a dose rate of 0.69 Gy/min. The animals will be immobilized during irradiation with ketamine (80 mg/kg), xylazine (5 mg/kg) and 0.4% isoflurane (100% O₂), or 2-3% isoflurane (100% O₂), to maintain treatment precision. The first group will receive a single dose of 5 Gy of ionizing radiation. The second group will receive a single dose of 10 Gy of radiation. The third group will receive a single dose of 15 Gy of radiation. The fourth group will receive 10×1 Gy with a 24 hour interval. The first five doses will be administered over a five day period, followed by a two day gap, and then the last five doses will be administered over another five day period. The fifth group will receive 10×2 Gy with a 24 hour interval. The first five doses will be administered over a five day period, followed by a two day gap, and then the last five doses will be administered over another five day period. The sixth group will receive 5×2 Gy with a 24 hour interval. The five doses will be administered over 5 days. The seventh group will not receive any radiation treatment and will serve as a control. After X-irradiation, the animals will be recovered and returned to standard housing.

Post-Irradiation Cognitive Testing.

The animals will be tested periodically after irradiation with the Morris Water Maze and Barnes Maze to ascertain if the radiation treatment altered memory and cognition, and whether any change in spatial learning and memory was temporary or permanent, or age related. The procedures for cognitive testing using the Morris Water Maze and Barnes Maze are well known in the art.

### Example 6

### Human Treatment and Evaluation Scheme

The following is a proposed treatment scheme for the treatment of AD with radiation in combination with neuro-cognitive testing and specialized diagnostic testing.

Treatment Scheme: One or more courses of treatment involving whole brain radiation, with a total dose per treatment course of 500-3000 cGy. The radiation will be given in 10-30 fractions of 50-200 cGy, with a dose rate of 400-600 Monitor Units per minute.

Treatment delivery: Ionizing radiation with photon energies between 4 and 6 MEV's will be delivered. Custom blocking will be employed to ensure that no doses are delivered to the anterior retina.

Diagnostic testing and neuro-cognitive studies will be performed prior to the initiation of the treatment, 3 months post-completion of treatment, and every 6 months thereafter for a period of 5 years.

Patient Inclusion:
1. Ages 30 to 100;
2. Life expectancy: At least 6 months
3. No previous partial or whole brain irradiation within the previous 5 years. Previous irradiation more than 5 years prior acceptable, so long as dose to hippocampal regions did not exceed 4500 cGy.
4. May not be pregnant.
5. The subject must be able to complete neuro-cognitive testing, such as a mini-mental state examination.
6. Subject must be able to be followed through the institution where the treatment is being delivered.

Exclusion Criteria:
1. Pregnancy
2. Previous history of partial or whole brain irradiation within previous 5 years.
3. Life expectancy less than 6 months
4. Inability to take neuro-cognitive testing

Treatment Scheme:
1. Evaluation by geriatric/neurology with diagnosis of early Alzheimer's disease.
2. Completion of neuropsychological testing evaluation including a Mini-mental state exam (MMSE).
3. Diagnostic imaging including positron emission tomography (PET) scan with Pittsburgh B-Compound and MRI evaluation.
4. Diagnostic cerebro-spinal fluid (CSF) cytology.
5. Evaluation and consultation by Radiation Oncologist.
6. Simulation using CT imaging.
7. Whole brain irradiation with custom blocks surrounding retinal surface.
8. 10-30 fractions of 50-200 cGy per fraction delivered over a 10-12 day period for a total dose of 500-3000 cGy.
9. Follow up at three months after the completion of treatment: Positron emission tomography (PET) scan with Pittsburgh B-Compound, MRI evaluation, and neurocognitive testing with mini-mental state exam.
10. Follow up at subsequent six-month intervals for the next five years: Positron emission tomography (PET) scan with Pittsburgh B-Compound, MRI evaluation, and neurocognitive testing with mini-mental state exam. If subject expires before the end of five years, a request for autopsy will be made especially related to the brain. P 11. Follow up at 3, 12, and 24 months after the completion of treatment: Cerebro-spinal fluid (CSF) cytology.

What is claimed is:

1. A method for treating dementia of the Alzheimer's type in a patient, the method comprising:
administering a therapeutically effective amount of ionizing radiation to the brain of the patient.

2. The method of claim 1, the radiation source is a helium, carbon, hydrogen, proton, neutron, electron, or photon source.

3. The method of claim 1, wherein the radiation is administered externally to the patient.

4. The method of claim 1, wherein the radiation is administered internally to the patient.

5. The method of claim 1, wherein the radiation is administered as an antibody labeled with a radionuclide.

6. The method of claim 1, wherein the radiation is administered in a total dose of 300-1800 cGy at 50% IDL (isodose line) by a focused based radiation source.

7. The method of claim 2, wherein the radiation is administered in a dose of 50 to 300 cGy per day by a linear accelerator.

8. The method of claim 2, wherein the radiation is administered in a dose of 50 to 600 cGy per day by a targeted radiation source.

9. The method of claim 2, wherein the radiation source is a gamma knife or cyber knife.

10. The method of claim 1, wherein the radiation is x-ray radiation.

11. The method of claim 1, wherein the radiation is gamma ray radiation.

12. The method of claim 2, wherein the radiation is targeted to the hippocampal region of the brain.

13. The method of claim 2, wherein the radiation is targeted to the frontal lobe of the brain.

14. A method for reducing the number or size of amyloid plaques or the extent of tau tangles in the brain of a patient, the method comprising: administering a therapeutically effective amount of ionizing radiation to the brain of the patient.

15. A method for inhibiting, arresting the development of, or preventing the progression of one or more clinical symptoms of Alzheimer's Disease in a patient, the method comprising: administering a therapeutically effective amount of ionizing radiation to the brain of the patient.
